(19) United States
(12) Patent Application Publication
(10) Pub. No.: US 2014/0200604 A1
(43) Pub. Date: Jul. 17, 2014

(54) FISTULA TREATMENT DEVICES AND METHODS

(71) Applicant: CURASEAL INC., Santa Clara, CA (US)

(72) Inventors: Harold F. CARRISON, Pleasanton, CA (US); Akshay MAVANI, Los Altos, CA (US); Kenton FONG, Mountain View, CA (US)

(73) Assignee: CURASEAL INC., Santa Clara, CA (US)

(21) Appl. No.: 14/155,593

(22) Filed: Jan. 15, 2014

Related U.S. Application Data
(60) Provisional application No. 61/752,910, filed on Jan. 15, 2013.

(51) Int. Cl. A61B 17/00; A61B 17/12; A61B 17/04

(52) U.S. Cl. CPC A61B 17/0057; A61B 17/0401; A61B 17/12031
USPC 606/191

(57) ABSTRACT

Disclosed herein are implantable fistula treatment devices and methods. In some embodiments, a distal anchor for an implantable fistula treatment device may comprise a suture and multiple foldable members including at least a distal-most foldable member and a proximal-most foldable member. The distal-most foldable member may comprise a suture attachment structure. The proximal-most foldable member may be configured to couple to a surface of a body lumen at a distal opening of a fistula.

### CROSS-REFERENCED TO RELATED APPLICATIONS

This application claims priority to U.S. Provisional Patent Application No. 61/752,910, filed on Jan. 15, 2014, entitled “Fistula Treatment Devices and Methods,” which is hereby fully incorporated by reference.

### TECHNICAL FIELD

The present invention relates to medical apparatus and methods. More specifically, the present invention relates to implantable devices for closing fistulas and methods of using such devices.

### BACKGROUND

Fistulas are a major cause of morbidity and mortality, as there are over one hundred thousand cases of pathologic fistulas a year, which account for over ten thousand deaths. They cost the healthcare system billions of dollars each year to treat.

Fistulas are tissue-lined connections between body cavities and hollow organs or between such cavities or organs and the surface of the body. The fistula tract includes a void or potential void in the soft tissues extending from a primary fistula opening to a blind ending or leading to one or more secondary fistula openings, sometimes following along tissue planes of organs or between organs. Fistulas frequently develop as a consequence of infections or accompany abscess formations. Although some fistulas are purposely created for therapeutic purposes such as tracheostomy tracts, gastric feeding tube tracts, or arteriovenous fistulas for dialysis access, pathological fistulas are abnormal tracts that typically occur either congenitally or form after surgery, surgery-related complications, or trauma. They are most often open tracts that have epithelialized, endothelialized, or mucosalized

Fistulas can form between almost any two-organ systems, or multiple organs between different sites of the same organ. For example, they may occur between internal organs and skin (enterocutaneous fistulas, gastrocutaneous fistulas, anal fistulas, rectovaginal fistulas, colocutaneous fistulas, vesiclocutaneous fistulas, intestinocutaneous fistulas, tracheocutaneous fistulas, bronchocutaneous fistulas, etc.) or between internal organs themselves (tracheal-esophageal fistulas, gastrointestinal fistulas, colovesicular fistulas, palatal fistulas, etc.). Fistulas may also form between blood vessels such as arteriovenous fistulas.

Fistulas may form in many locations in the body and are almost universally highly morbid to patients and difficult for clinicians to treat. For example, enterocutaneous fistulas are one of the most feared complications of abdominal surgery. Enterocutaneous fistulas are abnormal connections that form between the bowel and skin and can occur after abdominal surgery, after trauma, or as a complication of Crohn's disease. Some reports estimate that enterocutaneous fistulas may form in as many as 1% of patients that undergo major abdominal surgery. They often require months of supportive care and/or major abdominal surgery. The overall mortality rate for patients that develop enterocutaneous fistulas remains high at around 20%.

Current options for treatment of enterocutaneous fistulas include long-term conservative management or major surgery. In the conservative management option, patients are placed on restricted enteric intake and managed with parenteral nutritional support. Fistula leakage is controlled using a stoma bag. If fistula output is high, drains are sometimes placed to try to control the fistula output. The chance of spontaneous closure of a fistula under conservative management is relatively low—around 25%. If fistulas fail to spontaneously close with conservative management after five weeks of bowel rest, many surgeons advocate surgical treatment, though supportive care could continue indefinitely. Patients with open fistula tracts often have ongoing associated malnutrition and electrolyte imbalance issues, as well as chronic non-healing abdominal wounds.

The major surgery option is associated with a mortality rate near 30%. The surgery involves resection of the diseased intestinal segment, extirpation of the fistula, and debridement of the fistulous tract through the abdominal wall and subcutaneous tissue. This major abdominal surgery often requires blood transfusion and post-operative ICU admissions. As a result of chronic inflammation and having abdomens that have been previously operated on, these patients typically form dense adhesions and have highly friable tissues. In addition, these patients can be severely malnourished. These conditions make operations on enterocutaneous fistulas extremely difficult and dangerous. After surgery, the patient is placed on total parenteral nutrition (“TPN”) for several days before the patient can be weaned off TPN and slowly introduced to normal foods.

Other treatment options may include implantable devices designed to aid in the closure of the fistula. These devices, however, may cause adverse immunological reactions in patients, may allow leakage of fluid around them, or may migrate or become dislodged when the patient exerts himself, such as during exercise. Thus, there is a need for an implantable device for closing a fistula that reduces the chance of adverse immunological reactions, leakage of fluid through the fistula tract, and migration or dislodgement during use.

### DESCRIPTION OF THE RELATED ART

A number of fistula treatment devices and methods have been described previously by the assignees of the present application. For example, such fistula treatment devices and methods are described in U.S. Pat. Nos. 8,177,809, 8,206,416 and 8,221,451, U.S. Patent Application Pub. Nos. 2013/0006283 and 2012/0016412 and PCT Patent Application Pub. No. WO/2012/174468. All of the above references are hereby incorporated herein in their entirety and may be referred to herein generally as the “Incorporated References.” The present disclosure is directed to various new features, enhancements and embodiments of fistula treatment devices such as those described in the Incorporated References. None of the features, enhancements or embodiments described herein, however, is limited to, or by, any particular embodiment described in the Incorporated References

These and other aspects and embodiments will be described in further detail below, in reference to the attached drawing figures.

### BRIEF DESCRIPTION OF DRAWINGS

Certain preferred embodiments and modifications thereof will become apparent to those skilled in the art from the detailed description below having reference to the figures that follow.

FIGS. 1A and 1B are diagrammatic perspective views of an anchor member of a fistula treatment device, including multiple flexible disc members, according to one embodiment;

FIG. 2 is a side view of an anchor member of a fistula treatment device, including multiple flexible disc members, according to an alternative embodiment;

FIG. 3 is a side view of an anchor member of a fistula treatment device, including multiple flexible torus-shaped members, according to an alternative embodiment;

FIG. 4 is a top view of an anchor member of a fistula treatment device, including multiple flexible disc members having non-circular shapes, according to an alternative embodiment;

FIG. 5 is a side view of an anchor member of a fistula treatment device, including multiple flexible disc members and including surface features for adhering to tissue, according to one embodiment;

FIGS. 6A and 6B are side and bottom views, respectively, of a bottom flexible disc of an anchor member of a fistula treatment device, including surface features for adhering to tissue, according to an alternative embodiment;

FIG. 7 is a side view of a portion of an anchor member of a fistula treatment device, according to one embodiment;

FIGS. 8A-8C are side views of an anchor member of a fistula treatment device, including multiple flexible disc members and tissue traction features for adhering to and pulling together tissue, according to an alternative embodiment;

FIG. 9 is a side view of a covered stent fistula treatment device, including an extra sealing member, according to an alternative embodiment; and

FIGS. 10A and 10B are side views of a disc loading device for a fistula treatment device, according to two alternative embodiments.

### DETAILED DESCRIPTION

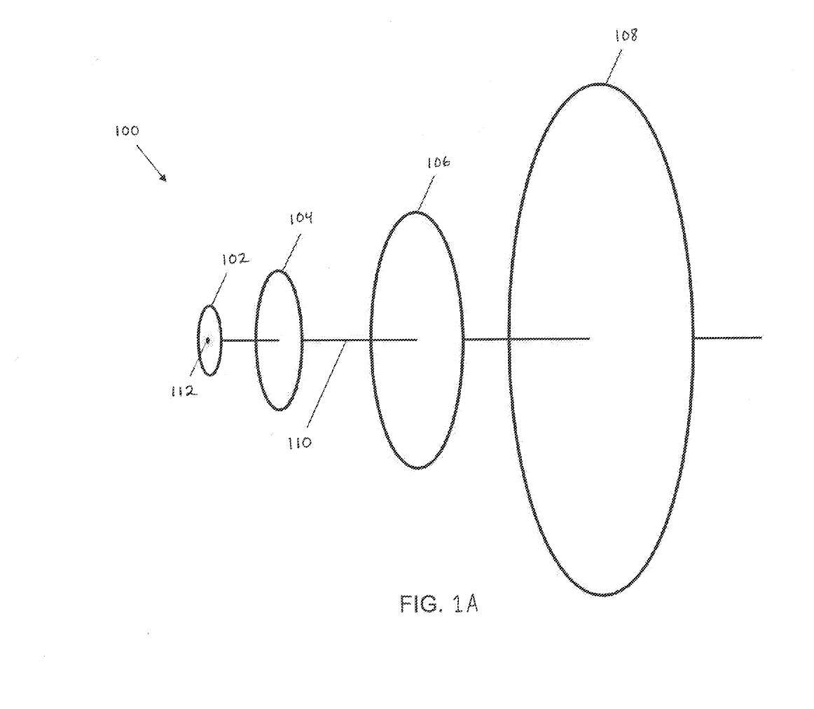

As described in the above-referenced Incorporated References, specifically, for example, in U.S. Patent Application Pub. Nos. 2013/0006283, in many embodiments, a fistula treatment device will include one or more anchoring members at one end. The anchoring members anchor the device within a body cavity at one end of the fistula, and in some embodiments part of the device extends from the anchor through the fistula. In one embodiment described previously, and as shown in FIGS. 1A and 1B, a distal anchor 100 for occluding a distal opening of fistula tract may include multiple foldable members 102, 104, 106, and 108 threaded on a suture 110. FIGS. 1A and 1B illustrate, respectively, an expanded and a restrained configuration of distal anchor 100. The expanded configuration illustrated in FIG. 1A may represent the configuration of the distal anchor 100 when it has been released from an insertion device into a body lumen. The restrained configuration illustrated in FIG. 1B may represent the configuration of the distal anchor when a restraining force is exerted on the distal anchor 100 by tensioning the suture 110 while the distal anchor 100 is positioned over a distal opening of a fistula tract.

In this application, the terms “proximal” and “distal” are used relative to a user of a device. In other words, the most distal portion of a device is the portion that is farthest from the user of the device when it is implemented, and the proximal portion is closest to the user when the device is implemented. In the case of the fistula treatment devices described herein, the distal end of a device is generally the end that is located deepest into the patient, and the proximal end is the end closest to the outside (skin) surface of the patient. In the multi-disc embodiment of FIGS. 1A and 1B, for example, the first foldable member 102 is the distal-most disc, and the last foldable member 108 is the proximal-most disc.

As can be appreciated by comparing FIGS. 1A and 1B, flexible members 104, 106, and 108 are configured to slide along suture 110. Proximal-most foldable member 108 may be further configured to occlude a distal opening of the fistula tract. Distal-most foldable member 102 may be configured to reduce or prevent rupturing at the center of foldable member 108 when the suture 110 is tensioned during positioning of the distal anchor 100. Distal-most foldable member 102 may be configured to a size and shape that distributes the force exerted by the suture over a wider area--the area of contact between foldable member 102 and the next foldable member, first inner foldable member 104. In this way, pressure exerted on foldable member 108 by tensioning suture 110 can be reduced. Inner foldable members 104 and 106 may also serve to reduce or prevent rupturing of the proximal-most foldable member 108 by further distributing the force exerted on foldable member 108. Distal-most foldable member 102 may also comprise a suture attachment structure 112 for attaching suture 110.

The embodiment shown in FIGS. 1A and 1B and many other embodiments of devices with flexible anchoring members are described in great detail in U.S. Patent Application Pub. Nos. 2013/0006283, previously incorporated by reference, so they will not be described again here.

In various alternative embodiments related to the one illustrated in FIGS. 1A and 1B, one or more disks positioned inside or outside the patient may be configured to create a pressure differential within the fistula, and this pressure differential may help to close the fistula. For example, in one embodiment, a disc that contacts the patient's skin on the outside of the body may have a default cupped (concave) shape, with the opening of the cup facing the patient's skin. If the cupped disc is forced onto the skin in a flattened shape and then released to resume its cupped shape, it will create a lower pressure in the fistula, compared to the pressure in the intestine, thus causing the fistula to partially or completely close. In another alternative embodiment, the entire bottom sealing member (or “disc”) may be cup shaped, so when it is held in place flat against the inner ostium wall with tension the device tries to again turn into a cup shape. This “yielding” of the device may provide two benefits: (1) It allows flexure of the fistula tract and the device will retain the seal as it flexes and takes up the different fistula tract length; and (2) It provides a slight decrease of pressure in the fistula tract.

In other alternative embodiments, disks may include textured surfaces for facilitating their joining together. Such surfaces may be similar to sand paper, for instance. In another embodiment, the disks may have interlocking features on the edges and main surfaces of the disks (i.e., the top and bottom surfaces).

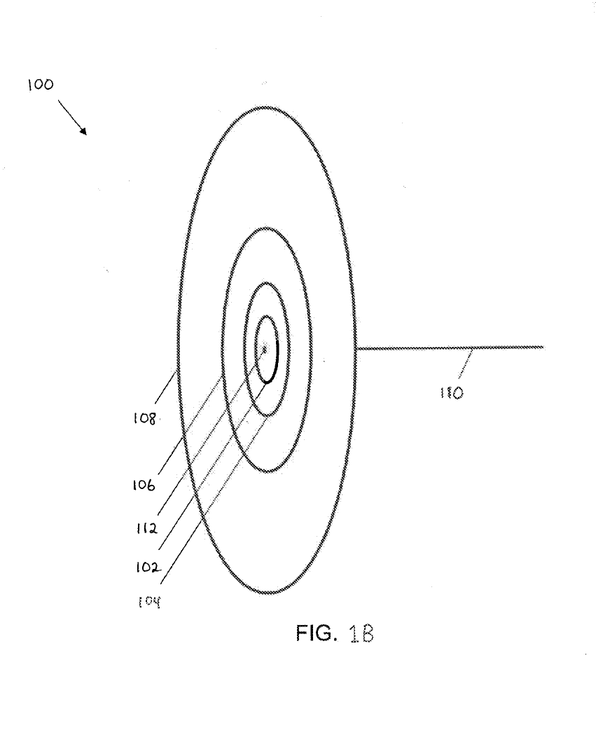

Also as described in U.S. Patent Application Pub. Nos. 2013/0006283, there are many suitable alternative embodiments of flexible anchoring members that include locking features to interlock with one another and thus prevent relative movement. Many examples are provided in the above-referenced patent application. In another alternative embodiment, and referring now to FIG. 2, an anchoring portion 200 of a fistula treatment device may include multiple layers, including a most-distal layer 210, a second layer 212, a third layer 214 and a most-proximal layer 216. In one embodiment, a protrusion 218 on the most-distal layer 210 may fit within apertures on the other layers 212, 214, 216. In various alternative embodiments, the protrusion 218 may extend all the way through or partway through the proximal-most layer 216, or alternatively the protrusion 218 may simply abut the top of the proximal-most layer 216.

In any embodiment that includes multiple flexible layers, such as but not limited to the embodiment shown in FIG. 2, at least one of the flexible layers may have a different thickness and/or a different stiffness than at least one other layer. In one embodiment, for example, a first layer (or proximal layer), which resides closest to the tissue adjacent the fistula, may be most flexible, a second layer may be stiffer, a third layer may be stiffer than the second layer, a fourth layer may be stiffer than the third layer, and so on. In an alternative embodiment, the opposite configuration may be used, with the first, proximal layer being stiffest and with subsequent layers being incrementally more flexible. Yet another alternative embodiment may include one layer having a first stiffness and all other layers having a second stiffness. In other alternative embodiments, a layer may have different stiffness within the layer itself, such as stiffer toward a middle portion and more flexible at an edge. Of course, multiple layers may have such variable stiffness as well. Any combination of stiffness/flexibility in the layers of an anchor member is possible, according to different embodiments.

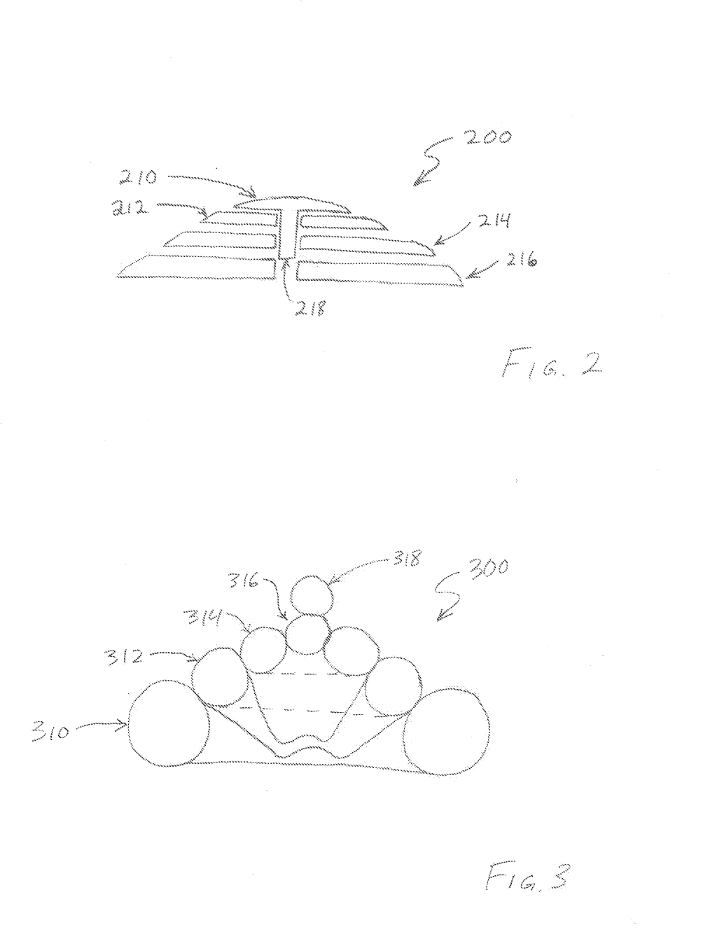

In another embodiment, and referring now to FIG. 3, an anchoring portion 300 of a fistula treatment device may include multiple stacking layers 310, 312, 314, 316, 318. At least some of the layers, starting with the bottom layer 310, may be shaped as a torus (i.e., doughnut-shaped). In the embodiment of FIG. 3, for example, layers 310, 312 and 314 are torus-shaped. One or more spherical layers 316, 318 may be disposed on top of the torus-shaped layers 310, 312, 314. When the layers are pulled downward from a topmost layer, for example via a suture or other pulling structure, the layers may form a seal with one another and with the tissue surrounding the opening of a fistula. In one alternative embodiment, a central post may be positioned within the circular openings of the torus-shaped layers, similar to a children's stacking toy, such that the torus-shaped layers fit over the post and form a seal.

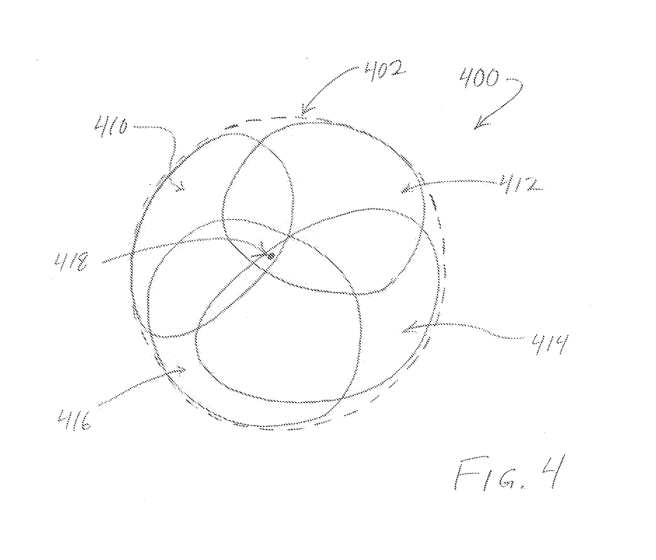

In some variations, and with reference now to FIG. 4, in some embodiments, an anchoring member 400 may include one or more foldable, flexible members 410, 412, 414, 416 that are non-circular. In one embodiment, the flexible members 410, 412, 414, 416 may all be smaller than an overall circumference 402 of the assembled anchoring member 400, and when assembled, the anchoring member 400 may approximate a circular shape 402. In one embodiment, the flexible members 410, 412, 414, 416 may be held together by an attachment member 418, such as a pin or suture. One advantage of using multiple, smaller-circumference layers to generate a larger circumference 402 is that each smaller layer 410, 412, 414, 416 may be easier to fold and advance through a small diameter delivery catheter through the fistula. In one embodiment, a very flexible, thinner layer (not pictured) with a circumference approximating that of circumference 402 may be positioned above or below the multiple smaller- circumference layers 410, 412, 414, 416, to facilitate assembly of the multiple layers or attachment.

A non-circular outline can be understood to be any shape in which the perimeter is not a constant radius from a center point. Non-circular shapes include shapes with first-derivative discontinuities at one or more locations. Non-circular shapes may also be a generally circular shape with protrusions or recesses on the perimeter to accommodate a predetermined surface of a body lumen. Non-circular shapes may include, but are not limited to, ovals, ellipses, rectangles, lenses, deltoids, and bell-shapes. When non-circular, a diameter of a foldable member may be understood to mean a length of the member in one dimension. For example, a line taken through a center point or a widest span of the member. In such variations, the diameters of the distal-most and inner foldable members may be characterized as a percentage from 1% to 100% of the diameter of the proximal-most foldable member, and may sometimes be about 5%, 10%, 20%, 25%, 30%, 35%, 40%, 45%, 50%, 55%, 60%, 65%, 70%, 75%, 80%, 85%, 90%, or 95%, or any percentage range between any two of the above percentages. In some variations, some of the foldable members take a shape different from one or more of the other foldable members. For example the distal members may be circular, but the proximal-most foldable member may be shaped to occlude a non-circular fistula opening. In some other variations, the distal foldable members are also non-circular in order to achieve a desired distribution of forces, for example.

In some variations, the proximal surface of the proximal-most foldable member may be structured to facilitate a secure and lasting coupling of the distal anchor to the surface of a body lumen. In some variations, the structure may be a tissue traction feature, as described herein. In some variations, an adhesive may be added to the proximal surface of the proximal-most member. The adhesive may be applied by a physician before inserting the proximal-most foldable member into the body lumen or applied after insertion. In other variations, the adhesive may be applied during a manufacturing process and covered with a liner. In some variations, the liner is removed by the physician prior to insertion. In other variations, the liner is configured to dissolve upon contact with bodily fluid or after a force is applied to the distal anchor. The adhesive may initially strengthen the bond of the proximal-most foldable member to the tissue and then gradually degrade in strength as fistula tract healing occurs or after fistula tract healing. Depending on the variation, the adhesive may create a fluid impermeable seal for at least 7, 14, 21, 28, 35, 60 or any other number of days.

In any of the embodiments described herein, all or a portion of an anchoring device or anchoring member of a fistula treatment device may be made of bioresorbable material. In one embodiment, all of the flexible members may be made of bioresorbable material. This is especially advantageous, because when an anchoring member is used to anchor a fistula treatment device within an intestine, for example, leaving a foreign body in the intestine for a long period of time may cause an intestinal blockage. If the anchoring member resorbs, this blockage risk is reduced. In some embodiments, one or more of the flexible members may contain or be coupled with a more permanent reinforcing structure, such as a wire mesh or metal film made of Nitinol or other suitable metal. In some cases, a thin reinforcing structure combined with an otherwise bioresorbable anchoring member may be beneficial.

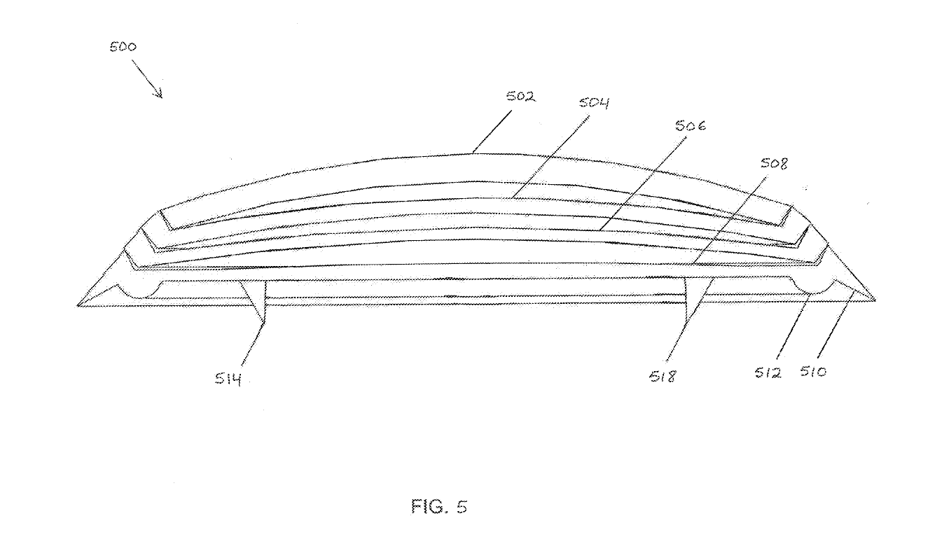

FIG. 5 depicts a cross-sectional view of a distal anchor 500, comprising distal-most foldable member 502, first inner foldable member 504, second inner foldable member 506, and proximal-most foldable member 508. In some variations, as depicted in FIG. 5, the distal-most foldable member 502, first inner foldable member 504, and second inner foldable member 506 may be curved. The proximal surface of proximal-most foldable member may be substantially planar. The distal surface of proximal-most foldable member 508 may comprise an outer region with a protrusion 512. Proximal-most foldable member 508 may also comprise a flat surface 510 connecting the edge of the proximal-most foldable member to protrusion 512. The proximal surface of proximal-most member 508 may also comprise tissue traction features 514 and 518 configured to engage the surface of a body lumen and restrain the distal anchor 500 with respect to the body lumen. As illustrated, tissue traction features 514 518 are typically located around the periphery of the proximal-most foldable member 508, leaving the inner portion of the member 508 smooth/flat. In some variations, one or more of tissue traction features 514 and 518 may be omitted. In other variations, additional tissue traction features are added. In various embodiments, tissue traction features 514 and 518 may have any suitable size and shape. As illustrated in FIG. 5, in one embodiment, the tissue traction features 514 and 518 have the shape of a bisected cone. In other embodiments, tissue traction features may have a cone shape, pyramidal shape, pointed rectangular shape, half-dome pointed shape or the like.

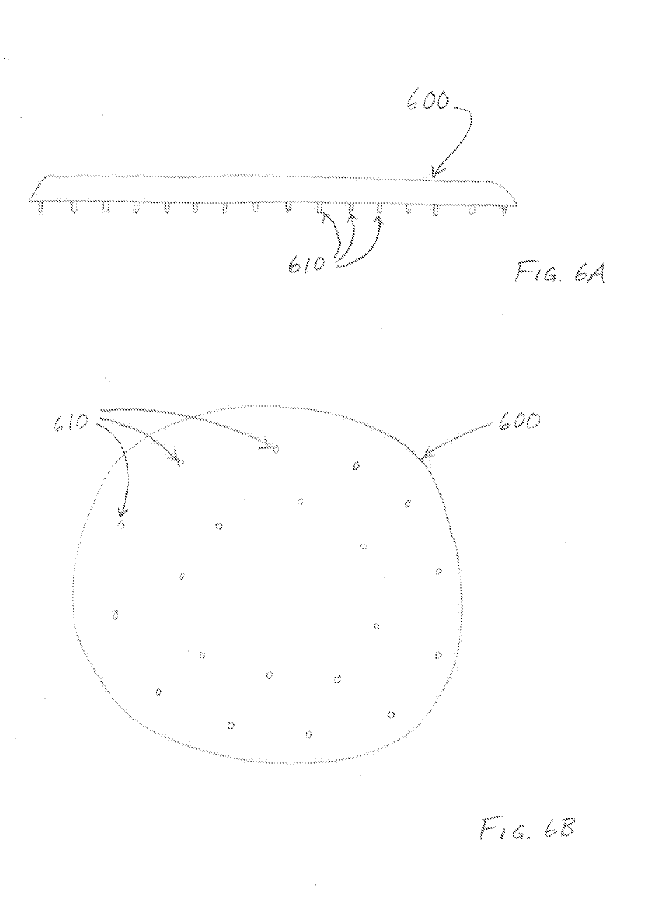

Referring now to FIGS. 6A and 6B, in another embodiment, a proximal layer 600 of an anchoring member may include multiple microneedles 610, such as pins, hooks and/or barbs. The microneedles 610 may be distributed throughout the proximal surface of the proximal-most member, but may also be distributed at predetermined locations. In some variations, the microneedles are distributed along a perimeter of the proximal surface, but in other variations the microneedles may be distributed at a position where contact is anticipated, such as the inner sealing regions described herein. The microneedles may be made of any suitable material, such as but not limited to Nitinol or a bioresorbable material.

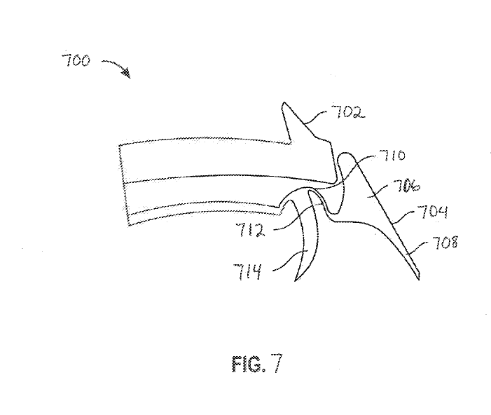

Referring now to FIG. 7, in one embodiment, an anchoring member 700 may include one or more curved tissue traction features 714 (or “prongs”) configured to partially or completely penetrate tissue and close an opening in the tissue. FIG. 7 depicts a portion of a distal anchor 700 comprising inner foldable member 702 and proximal-most foldable member 704. Inner foldable member 702 may comprise a geometry similar to any of the inner foldable members described herein. Proximal-most foldable member 704 may comprise a distal protrusion 706 and outer region 708. Distal protrusion 706 may comprise a geometry similar to any of the protrusions described herein. Outer region 708 may comprise a geometry similar to any of the outer regions of the proximal-most foldable members described herein. Proximal-most foldable member 704 also comprises a moveable protrusion 710 on its distal surface, a recess 712 on its proximal surface, and a tissue traction feature 714 on its proximal surface. Moveable protrusion 710 and recess 712 may be aligned to create a region of reduced thickness in proximal-most foldable member 704. Recess 712 and tissue traction feature 714 may be interconnected so that tissue traction feature 714 enters and grips the tissue of a body lumen as inner foldable member 702 connects with proximal-most foldable member 704. More specifically, as the proximal surface of inner foldable member 702 engages with moveable protrusion 710, the protrusion is forced proximally, thereby forcing distal recess 712 proximally. Distal recess 712 and tissue traction feature 714 may be integrally coupled so that tissue traction feature 714 moves proximally and inwardly as distal recess 712 moves proximally. In this way, the proximal motion of inner foldable member 702 is translated to a proximal and inward motion of tissue traction feature 714, thereby facilitating entering and gripping of the tissue.

Protrusion 710 is depicted as circular, but in some variations protrusion 710 is non-circular. When circular, protrusion 710 might be characterized as an arc with a radius that intersects the distal surface of an inner region of proximal-most foldable member 704. In some variations, the radius of the arc is described as a percentage of the diameter of the proximal-most foldable member and may sometimes be 1%, 2%, 3%, 4%, 5%, 10%, 15%, 20%, 25%, 30%, or any percentage range between any two of the above percentages. In some variations, the arc does not have a constant radius. In some variations, protrusion 710 may be less resistant to movement than surrounding areas of the proximal-most foldable member 704. In this way, protrusion 710 may be configured to move relative to the surrounding area of proximal-most foldable member. In some variations, the reduction in resistance to deformation is facilitated by a decrease in the thickness of the proximal-most foldable member 704 in the area of the protrusion 710. In other areas, the density of the material is reduced in the area of the protrusion 710. Although FIG. 7A depicts proximal-most foldable member 704 as comprising a single protrusion configured to move relative to the surrounding area, other variations may have any number of such protrusions, including 2, 3, 4, 5, 6, 7, 8, and 10 protrusion. Further, FIG. 7A illustrates a protrusion on the distal surface of proximal-most foldable member 702, but some variations may include a protrusion on the proximal surface of inner foldable member 702 and a flat surface or protrusion on the distal surface of proximal-most foldable member 704.

Tissue traction feature 714 is illustrated as being “fang” shaped, but in other embodiments tissue traction feature 714 takes an alternative shape, such as a hook shape, that can puncture the surface of a body lumen. Tissue traction feature 714 may comprise barbs oriented away from the direction of insertion, thereby preventing withdrawal of the fang after insertion. In some variations, the length of tissue traction feature 714 is described as a percentage of the thickness of proximal-most foldable member 704 from its distal-most point to its proximal-most point, and the percentage may sometimes be 5%, 10%, 20%, 30%, 40%, 45%, 50%, 60%, 70%, 80%, 90%, or 95%, or any percentage range between any two of the above percentages. In other variations, the thickness of proximal-most foldable member 704 from its distal-most point to its proximal-most point is described as a percentage of the length of tissue traction feature 714, and the percentage may sometimes be 5%, 10%, 20%, 30%, 40%, 45%, 50%, 60%, 70%, 80%, 90%, or 95%, or any percentage range between any two of the above percentages.

Although FIG. 7 illustrates protrusion 710, recess 712, and tissue fraction feature 714 positioned near an edge of foldable member 704, other variations may have the tissue traction feature positioned at any location on proximal-most foldable member 704. In some variations, the position of the protrusion 710, recess 712, and tissue traction feature 714 is characterized as a percentage of the radius of the proximal-most member and may sometimes be 5%, 10%, 20%, 30%, 40%, 45%, 50%, 60%, 70%, 80%, 90%, or 95%, or any percentage range between any two of the above percentages. Further, although portion 700 is described with an inner foldable member, a distal-most foldable member may replace inner foldable member 702 without deviating from the scope of the disclosure.

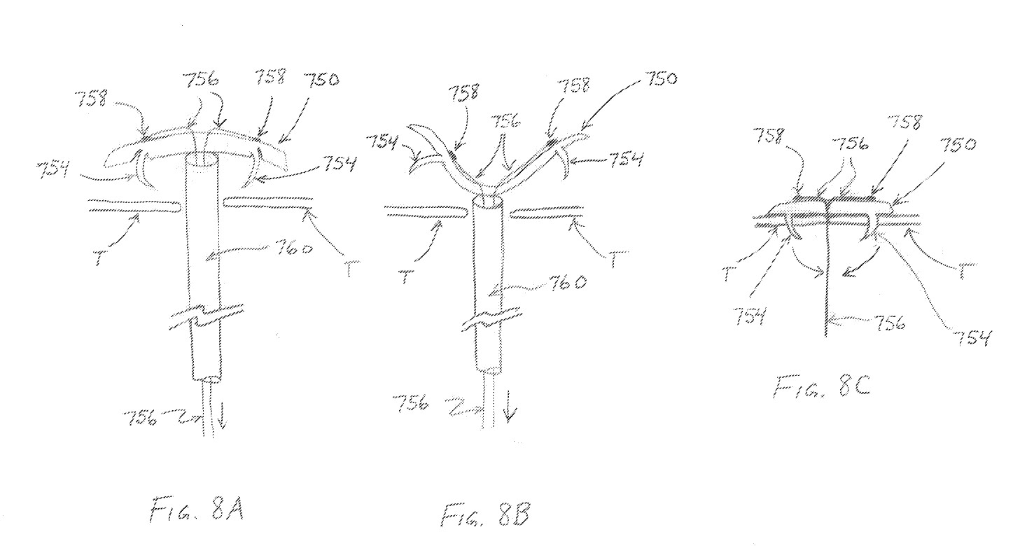

FIGS. 8A-8C illustrate one embodiment of a method for attaching the distal anchor 750 to tissue T in a way that an opening in the tissue T, such as tissue forming an opening of a fistula, is closed. As illustrated in FIG. 8A, as a first step, a catheter 760 delivery device may be positioned to abut the anchor 750. Suture 756 may be attached to the anchor 750 at attachment points 758 and may extend through the catheter 760. The suture 756 may be pulled from an opposite end of the catheter 760, as illustrated by the arrow in FIGS. 8A and 8B, thus placing tension on the anchor 750 and causing it to deform upward at its edges, as illustrated in FIG. 8B. Finally, as shown in FIG. 8C, the anchor 750 may be pulled down onto the tissue T, causing the tissue traction features 754 to enter the tissue along a curved path and thus pull opposing edges of the tissue T together to close the opening. Thus, the tissue traction features 754 not only attach the anchor 750 to the tissue T, but also bring the tissue edges together. Such an embodiment might be especially useful for treatment of enteroatmospheric fistulas, in which the diameter of the opening in the tissue is large and the length of the fistula is short.

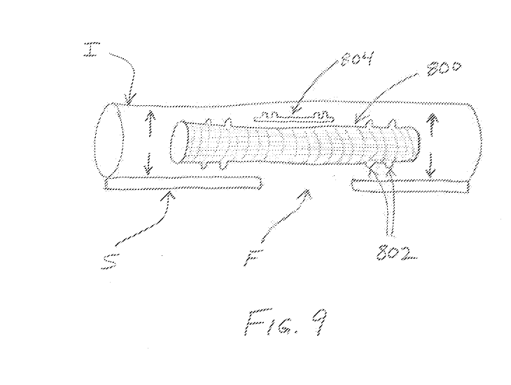

Referring now to FIG. 9, as explained more fully in the Incorporated References, an enteroatmospheric fistula typically has a relatively large diameter relative to the length of the fistula itself In some case, for example, the length of the fistula is merely the thickness of a layer of skin and subdermal tissue. The example shown in FIG. 9 diagrammatically illustrates a portion of an intestine I, with adjacent skin S and an enteroatmospheric fistula F. One way of treating such a fistula F, as described in the Incorporated References, is to place a covered stent 800 in the intestine I (or other body lumen or cavity in alternative embodiments) and expand the stent 800 (arrows in FIG. 9) to form a seal between the stent 800 and the intestine. It is very important, in such embodiments, to form a good seal between the stent 800 and the intestine I. To that end, in some embodiments, the stent 800 may include sealing members 802 at or near both ends of the stent 800. Additionally, in some embodiments an extra, separate sealing device 804 may be attached to the stent 800 (via adhesive or other means) to enhance/strengthen the seal between the stent 800 and the intestine I. Such a sealing device 804 may be circumferential or partially circumferential, and in various embodiments any number of separate sealing devices 804 may be used.

The stent 800 may be delivered into the intestine I (or other body lumen or cavity in alternative embodiments) using any suitable method. In some embodiments, for example, the stent 800 is delivered into the lumen through the fistula F, either by directly passage through the fistula F or via a tubular delivery device advanced into the fistula F. When the stent 800 is delivered through the fistula F, it must be pulled back within the intestine I so that it crosses the intestinal opening of the fistula F. The stent 800 must also be expanded to form the seals with the intestine I. In various embodiments, a method for delivering the stent 800 into the intestine I or other body lumen/cavity may be performed by: (1) inserting the stent 800, pulling the stent 800 back, and expanding the stent 800; (2) inserting the stent 800, partially expanding the stent 800, pulling the stent 800, and fully expanding the stent 880; (3) inserting a multi-segment stent (not pictured) in pieces, assembling the stent, and expanding the stent; or (4) inserting a multi-segment stent in pieces, partially expanding the stent, assembling the stent, and fully expanding the stent. Any combination or order of such steps is contemplated with the scope of the invention, according to various embodiments.

In one alternative embodiment, a stent may be bendable in the middle or near the middle, to allow it to be advanced through the fistula F in a bent configuration. This bendable stent would then straighten, after delivery into the intestine I or other body lumen or cavity. Such a bendable stent made be made of Nitinol or other shape memory material, so that it resumes its default, straight configuration upon delivery.

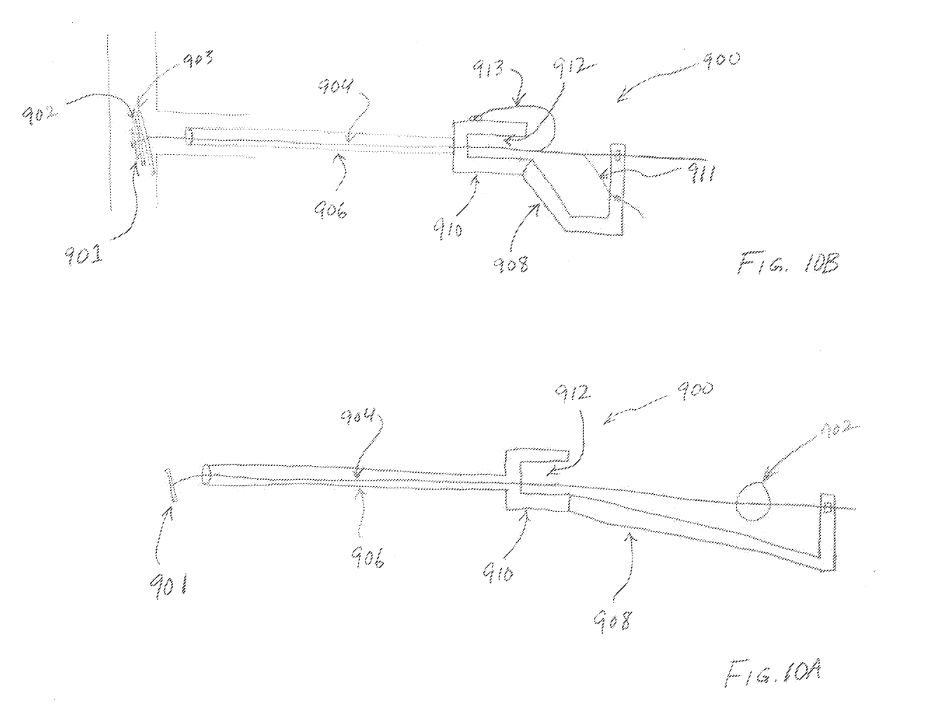

Referring now to FIG. 10A, one embodiment of a loading device 900 for an enterocutaneous fistula treatment device is illustrated. The loading device 900 may include a catheter 906 (or “tubular member”) attached proximally to a hub 910, which in turn is coupled with a handle 908. The hub 910 may include a slot 912. The loading device 900 may be used to advance multiple, flexible anchoring members 901, 902 (or “disks”) over a suture 904, which is held in place by the handle 908. In some embodiments, the disks 901, 902 may be advanced over the suture 904 by advancing them one-by-one into the hub 910 and down the catheter 906. In one embodiment of the method, a user may hold a disk 902 between two fingers. The disk 902 and the fingers may then be advanced into the slotted hub 910 so that the disk 902 is approximately symmetrically sticking out of the slots. The fingers may then be rotated in the hub 910 so the disk 902 rolls up around the fingers. Next, the fingers may be more fully inserted down the hole in the hub 910 and into the catheter 906.

In some embodiments, insertion may also involve the use of a rod (not illustrated), which may include a slot in it for the disks 902, is relatively small in diameter, such as about 0.080″ in one embodiment, and may have a slot that is about 0.025″ wide and about 0.75″ long, according to one embodiment. A disk 902 may be placed into the slot so that the rod bisects the disk 902. The rod may then be placed into the hub 910 until the disk bottoms out on the slot in the hub 910. The rod is then turned, and the disk 902 rotates around the rod. When the disk 902 is fully wrapped upon the rod, the rod may be more fully inserted into the hub 910 and into the catheter 906. A tube over the rod and located proximally to the disk slot 912 is used to push the disk 902 off the rod when it is placed into the catheter 906.

The disk 901 that is first placed into the patient (the distal-most disk) has the suture 904 move with the disk 901. The second disk 902 slides over the suture 904 as it is advanced into the hub 910 and down the catheter 906. This requires the suture 904 to be held fixed in relationship to the catheter 906. The handle 908 allows the suture 904 to be held in place while the disks 902 are advanced down the suture 904. This feature allows disks 901, 902 to be advanced down the suture 904 without requiring the user to have an assistant perform part of the loading process.

Referring now to FIG. 10B, in an alternative embodiment, the loading device 900 may include a more curved handle 908. Also, in this embodiment, the loading device 900 includes attachment points for additional sutures 911, 913, as in some cases it may be advantageous to use multiple sutures for loading disks 901, 902, 903. In all other respects, the embodiment of FIG. 10B is the same as that of FIG. 10A.

Numerous changes, variations, and substitutions will occur to those skilled in the art without departing from the invention. Various alternatives to the embodiments of the invention described herein may be employed in practicing the invention.

What is claimed is:

1. A distal anchor for an implantable fistula treatment device, the distal anchor comprising:
a suture;
a distal-most foldable member having a proximal-facing protrusion;
a suture attachment structure attaching the suture to the distal-most foldable member; and
multiple additional foldable members, including a proximal-most foldable member and at least one additional foldable member positioned between the distal-most foldable member and the proximal-most foldable member,
wherein the multiple additional foldable members are configured to slide along the suture toward the distal-most foldable member, and wherein at least some of the additional foldable members include an aperture sized to accept the proximal-facing protrusion of the distal-most foldable member to lock the foldable members in position relative to the distal-most foldable member,
wherein the proximal-most foldable member is configured to couple to a surface of a body lumen at a distal opening of a fistula and occlude the fistula at the distal opening.

2. The distal anchor of claim 1, wherein a diameter of the distal-most foldable member is smaller than a diameter of any of the multiple additional foldable members, and wherein a diameter of the proximal-most foldable member is greater than a diameter of the distal-most foldable member or of any of the other additional foldable members.

3. The distal anchor of claim 1, further comprising multiple tissue traction features disposed on a tissue contacting surface of the proximal-most foldable member.

4. The distal anchor of claim 3, wherein the tissue traction features comprise microneedles.

5. The distal anchor of claim 1, wherein at least some of the foldable members are bioresorbable.

6. The distal anchor of claim 1, wherein the distal-most foldable member is pre-attached to the suture via the suture attachment structure.

7. The distal anchor of claim 6, wherein the additional foldable members are not pre-attached to the suture.

8. A distal anchor for an implantable fistula treatment device, the distal anchor comprising:
a suture;
a distal-most member;
a suture attachment structure attaching the suture to the distal-most member; and
multiple torus-shaped foldable members, including a proximal-most torus-shaped foldable member and at least one additional torus-shaped foldable member positioned between the distal-most member and the proximal-most foldable member, wherein the multiple foldable members are configured to slide along the suture toward the distal-most member, and wherein at least one of the multiple foldable members forms a seal with the distal-most member or another foldable member adjacent the distal-most foldable member,
wherein the proximal-most foldable member is configured to couple to a surface of a body lumen at a distal opening of a fistula and occlude the fistula at the distal opening.

9. The distal anchor of claim 8, wherein each of the torus-shaped foldable members includes at least one locking feature configured to lock with a corresponding locking feature on an adjacent one of the torus-shaped foldable members.

10. The distal anchor of claim 8, further comprising a post for extending through central openings of the multiple torus-shaped foldable members.

11. The distal anchor of claim 8, wherein the distal-most member comprises a sphere.

12. The distal anchor of claim 11, further comprising an additional spheroidal member disposed between the distal-most member and a most-distal of the torus-shaped foldable members.

13. The distal anchor of claim 8, wherein the distal-most member is foldable.

14. The distal anchor of claim 8, wherein at least some of the foldable members are bioresorbable.

15. The distal anchor of claim 8, wherein the distal-most member is pre-attached to the suture via the suture attachment structure.

16. The distal anchor of claim 15, wherein the foldable members are not pre-attached to the suture.

17. A distal anchor for an implantable fistula treatment device, the distal anchor comprising:
a suture;
a distal-most foldable member;
a suture attachment structure attaching the suture to the distal-most foldable member; and
multiple additional foldable members, including a proximal-most foldable member and at least one additional foldable member positioned between the distal-most foldable member and the proximal-most foldable member, wherein the multiple additional foldable members are configured to slide along the suture toward the distal-most foldable member,
wherein each of the distal-most foldable member and the multiple additional foldable members is non-circular and has a circumference that is smaller than an overall circumference of the distal anchor when it is assembled,
wherein the distal anchor, when assembled, has an approximately circular shape, and
wherein the proximal-most foldable member and at least one other of the foldable members, when joined together, are configured to couple to a surface of a body lumen at a distal opening of a fistula and occlude the fistula at the distal opening.

18. The distal anchor of claim 17, wherein at least some of the foldable members are bioresorbable.

19. The distal anchor of claim 17, further comprising an adhesive on a tissue contacting surface of at least the proximal-most foldable member.

20. The distal anchor of claim 17, further comprising multiple tissue fraction features disposed on a tissue contacting surface of the proximal-most foldable member.

21. The distal anchor of claim 17, wherein the tissue traction features comprise microneedles.

22. The distal anchor of claim 17, wherein the distal-most foldable member is pre-attached to the suture via the suture attachment structure.

23. The distal anchor of claim 22, wherein the additional foldable members are not pre-attached to the suture.

24. A distal anchor system for an implantable fistula treatment device, the distal anchor system comprising:
a distal anchor, comprising:
multiple sutures;
a tissue contacting foldable member including multiple tissue traction prongs on a tissue facing, proximal side; and
a suture attachment structure attaching the sutures to the foldable member; and
a delivery catheter through which the distal anchor is delivered through a fistula,
wherein, after delivery of the foldable member out of a distal end of the delivery catheter, upon pulling back on the sutures while leaving the catheter in place, thus placing the sutures in tension, a perimeter of the foldable member is pulled upward by the sutures at their attachment points via the suture attachment structure, and
wherein, when the tension on the sutures is relieved, the perimeter of the foldable member moves toward tissue surrounding the fistula, causing the prongs of the tissue contacting foldable member to engage the tissue and reduce a diameter of the fistula.

25. The system of claim 24, further comprising additional foldable members that, together with the tissue contacting foldable member, form the distal anchor.

26. The system of claim 25, wherein at least some of the foldable members are slidable along the sutures.

27. The system of claim 25, wherein at least some of the foldable members are bioresorbable.

28. A method of sealing a fistula tract, the method comprising:
advancing a flexible distal anchor through a distal end of a catheter and through an opening of the fistula tract;
pulling back on multiple sutures connected to the distal anchor at or near an outer edge of the anchor, while maintaining the catheter relatively stationary relative to the distal anchor, wherein the multiple sutures extend through a central portion of the distal anchor and through the catheter, and wherein pulling back on the multiple sutures places the sutures under tension and causes the outer edge of the anchor to rise above tissue surrounding the opening of the fistula tract; and
relieving at least some of the tension applied to the sutures to allow the outer edge of the anchor to move toward the tissue surrounding the opening, thus causing at least two curved prongs on a tissue-facing surface of the distal anchor to engage the tissue and reduce a diameter of the opening of the fistula tract.
